(19) United States
(12) Patent Application Publication
(10) Pub. No.: US 2024/0139648 A1
(43) Pub. Date: May 2, 2024

(54) A PROCESS FOR EXTRACTION OF BIOMOLECULES FROM BIOMASS

(71) Applicant: COUNCIL OF SCIENTIFIC AND INDUSTRIAL RESEARCH, New Delhi (IN)

(72) Inventors: Raja THIRUMALAISWAMY, Maharashtra Pune (IN); Vipul SUBHASH PATIL, Maharashtra Pune (IN); Yogesh ASHOK MAHAJAN, Maharashtra Pune (IN); Samruddhi AJIT MANE, Maharashtra Pune (IN); Nikitra NIHAL CHAND GUPTA, Maharashtra Pune (IN); Akash RAVINDRA BHATKAR, Maharashtra Pune (IN); Mekala SIVA PRASAD, Maharashtra Pune (IN)

(21) Appl. No.: 18/272,851

(22) PCT Filed: Jan. 20, 2022
(86) PCT No.: PCT/IN2022/050043

§ 371 Date: Jul. 18, 2023

(30) Foreign Application Priority Data
Jan. 20, 2021 (IN) 202111002836

(51) Int. Cl. B01D 11/02

(52) U.S. Cl. CPC B01D 11/0265; B01D 11/0284

(57) ABSTRACT

The present invention discloses a process for extracting biomolecules from biomass using an extraction apparatus. Further, the present invention provides an ultrasonic process that extracts lutein pigments from marigolds using an extraction apparatus that requires at least two repeating units of reactor setup. Here the present invention shows an extraction efficiency in the range of 95-100% and purity of biomolecules with a range of 90-95% under mild reaction conditions.

### FIELD OF THE INVENTION

The invention relates to a continuous process for extracting biomolecules from biomass by using an extraction apparatus. Particularly, the invention discloses an ultrasonic process for extracting lutein pigments from marigold using an extraction apparatus that requires at least two repeating units of reactor set-up.

### BACKGROUND AND PRIOR ART OF THE INVENTION

Lutein is one such important member of the carotenoid (xanthophyll) family widely present in marigold flower, spinach; kale used as a functional food as it has vivid applications from poultry to ophthalmic-pharmacy industry. Lutein and its isomer zeaxanthin are the oxidation products of carotenoids in plants. Both lutein and its stereoisomer zeaxanthin are distinguished from other carotenoid compounds based on the chemical composition of hydroxyl group attachments to their structures.

Lutein and its sister isomer zeaxanthin are the only macular pigments that prevent UV damage by the virtue of their rich antioxidant activity. Its antioxidant activity is to protect the outer retina, which is rich in polyunsaturated fats, from light-induced free radicals. Animals do not possess the potential to synthesize those pigments naturally. Different extraction techniques for lutein pigment from various biomasses have been proposed and demonstrated by numerous authors.

US patent application No. US20100305366 discloses an ultrasonic lutein extraction process, which avoids the adverse influence of solvent extraction on lutein stability, improves the extraction speed of lutein, and increases the extraction rate of effective components. Here, the ultrasonic extraction method utilizes ultrasonic wave-induced intensive vibration, high acceleration, intensive cavitation effect, and stirring action to accelerate the entrance of lutein into a solvent, to increase the extraction rate of effective components and shorten the extraction time to extract lutein with a purity of up to 95%.

Indian patent Application No. 201641017567 discloses an ultrasonication extraction process for the production of lutein from microalgal biomass using a minimal amount of permissible organic solvents. Here, the extraction process extracts lutein with a purity of 90%.

Chinese patent application No. CN110746332 discloses a method of extracting lutein and nostoc composite powder from marigold flowers and nostoc commune using an ultrasonic-assisted extraction process.

However, most of the lutein extraction processes show an extraction efficiency of between 90-95%, which causes the phenomenon of raw material waste. Till this time, there is no extraction process available in the market that can extract lutein without any raw material waste. So there is a need in the art to develop an extraction process that can overcome this drawback.

Accordingly, the present invention provides a continuous process for extracting lutein pigment from marigold having an extraction efficiency of 100% and a purity of 95%.

### OBJECTIVES OF THE INVENTION

The main objective of the present invention is to provide a process of extraction of biomolecules from biomass by using an extraction apparatus. Consequently, another object of the present invention is to provide an ultrasonic extraction process for extracting lutein pigments from marigolds using an extraction apparatus that requires at least two repeating units of reactor setup.

### SUMMARY OF THE INVENTION

Accordingly, the present invention discloses a process of extracting biomolecules from biomass using an extraction apparatus.

In an embodiment of the present invention, the continuous process for extracting biomolecules from biomass comprises the steps of:
(a) introducing biomass into the first extractor (1), from the top inlet hopper followed by interacting the bio-mass with the extraction solvent, coming from the bottom of the extractor wherein the extraction solvent dissolves the biomass's valuable biomolecules;
(b) collecting the extracted biomolecules at the first phase separator (4) and recycling the extraction solvent to the feed tank;
(c) treating the spent solids from the first extractor (1) with co-solvent in the first interstage processing vessel (19) in the influence of ultrasonic cavitation;
(d) pumping the partially processed biomass into the second extractor (2), from the top inlet followed by interacting with extraction solvent which is coming from the bottom of the extractor wherein the extraction solvent dissolves the valuable biomolecules from the bio-mass;
(e) collecting the extracted biomolecules at the second phase separator (5) and the extraction solvent is condensed and sent to the first extractor (1);
(f) treating the spent solids from the second extractor (2) with co-solvent in the second interstage processing vessel (20) in the influence of ultrasonic cavitation;
(g) pumping the partially processed biomass from the second extractor (2) into the third extractor (3) from the top inlet followed by interacting with the extraction solvent, coming from the bottom of the third extractor (3) wherein the extraction solvent dissolves the biomass's valuable biomolecules and
(h) collecting the extracted biomolecules at the third phase separator (6) and the extraction solvent is condensed and sent to the second extractor (2);
wherein the spent solids from third extractor are treated as the spent solid mass.

In another embodiment of the present invention, the extraction efficiency of the process is in the range of 95-100%

In still another embodiment of the present invention, the purity of extracted biomolecules is in the range of 90-95%.

In another embodiment of the present invention, the biomolecules are selected from lutein, astaxanthin, colchicines, citral, citronellal and other major flavors and fragrances.

In a preferred embodiment of the present invention, thebiomolecule is lutein.

In an embodiment of the present invention, the biomass is selected from yellow and brownish flower like marigold, maize, glory lily, algae like Haematococcus pluvialis, Chlorella zofingiensis, Chlorococcum, and Phaffiarhodozyma, some plants like lemon myrtle, Litseacitrata, Litseacubeba, lemongrass, lemontea-tree, Ocimum gratissimum, Lindera citriodora, Calypranthes parriculata, petitgrain, lemon verbena, lemon ironbark, lemon balm, lime, lemon and orange, ashwagandhaa, etc

In a preferred embodiment of the present invention, the biomass is marigold.

In an embodiment of the present invention, the ultrasonic process of extracting lutein pigments from marigold is done using an extraction apparatus that requires at least two repeating units of reactor set-up.

In a preferred embodiment of the present invention, the extraction apparatus is a reactor.

In an embodiment of the present invention, the reactor comprising of:
a) Extractor (1-3)
b) Phase separator (4-6)
c) Rotary valve (7-9)
d) Pump (10-12)
e) Cavitation Probe (13-15)
f) Condenser (16-18)
g) Interstage processing Vessel (19-21)
h) Solvent Feed vessel (22)
i) Product collectors (23-25)
j) Pressure regulating valve (26-28)
k) Vent valves (29-31)
l) Heating Jacket (32-34)
m) Co-solvent (35-37)
n) Pump (38)
o) Feed inlet (39)
p) Solvent (40)
q) Spent solidFeed (41)

In another embodiment of the present invention, the extraction apparatus for extraction of biomolecules from biomass comprising:
(a) extractors including a first extractor (1), a second reactor (2) and a third reactor (3) to extract biomolecules from the biomass when the biomass interacts with an extraction solvent (40);
(b) phase separators including a first phase separator (4), a second phase separator (5), and a third phase separator (6) to provide a required latent heat of evaporation to separate the solvent from extracted products;
(c) interstage processing vessels including a first interstage processing vessel (19), a second interstage processing vessel (20), and a third interstage processing vessel (21) to collect the under processed solid biomass;
(d) cavitation probes including a first cavitation probe (13), a second cavitation probe (14), and a third cavitation probe (15) to induce the desired ultrasonic cavitation effect into the interstage processing vessels (19,20,21) respectively; and
(e) condensers including a first condenser (16), a second condenser (17), and a third condenser (18) to condense and recycle the solvent (40) and use the recycled solvent as feed for the next reactor.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1: depict the reactor set up

FIG. 2: depict the lutein HPLC Analysis Chromatograph

FIG. 3: depict the colchicine analysis HPLC-MS Chromatograph

### DETAILED DESCRIPTION OF THE INVENTION

While the invention has been disclosed with reference to certain embodiments, it will be understood by those skilled in the art that various changes may be made and equivalents may be substituted without departing from the scope of the invention. In addition, many modifications may be made to adapt to a particular situation or material to the teachings of the invention without departing from its scope.

Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein.

The tables, figures and protocols have been represented where appropriate by conventional representations in the drawings, showing only those specific details that are pertinent to understanding the embodiments of the present invention so as not to obscure the disclosure with details that will be readily apparent to those of ordinary skill in the art having benefit of the description herein.

As used herein, the terms “continuous process”, when used in the context of the present invention refers to any process in which the product comes out without interruption and not in groups.

As used herein, the terms “Sono-chemically controlled extraction” and “ultrasonic extraction process” are used interchangeably and refer to an extraction process using vibrations having an ultrasonic frequency.

Accordingly, to accomplish the objectives of the present invention, the inventors propose a continuous process for extracting biomolecules from biomass using an extractor.

In an embodiment of the present invention, the continuous process for extracting biomolecules from biomass comprises the steps of:
a) introducing a bio mass into the first extractor (1) from the top inlet hopper followed by interacting the bio-mass with the extraction solvent, coming from the bottom of the extractor wherein the extraction solvent dissolves the biomass's valuable biomolecules;
b) collecting the extracted biomolecules at the first phase separator (4) and recycling the extraction solvent to the feed tank;
c) treating the spent solids from the first extractor (1) with co-solvent in the first interstage processing vessel (19) in the influence of ultrasonic cavitation;
d) pumping the partially processed biomass into the second extractor (2) from the top inlet followed by interacting with extraction solvent which is coming from the bottom of the second extractor (2) wherein the extraction solvent dissolves the valuable biomolecules from the bio-mass;
e) collecting the extracted biomolecules at the second phase separator (5) and the extraction solvent is condensed and sent to the first extractor (1);
f) treating the spent solids from the second extractor (2) with co-solvent in the second interstage processing vessel (20) in the influence of ultrasonic cavitation;
g) pumping the partially processed biomass from the second extractor (2) into the third extractor (3) from the top inlet followed by interacting with the extraction solvent, coming from the bottom of the third extractor (3) wherein the extraction solvent dissolves the biomass's valuable biomolecules and
h) collecting the extracted biomolecules at the third phase separator (6), and the extraction solvent is condensed and sent to the second extractor (2);
wherein, the spent solids from the third extractor (3) is treated as the spent solid mass.

In another embodiment of the present invention, the retention time of solids in each extractor is 10-20 min.

In still another embodiment of the present invention, the extraction process is carried out at a temperature ranging from 20-45° C.

In yet another embodiment of the present invention, the operation pressure for extractor 3, extractor 2 and extractor 1 are 30-40 bar, 25-30 bar and 15-25 bar respectively.

In an embodiment of the present invention, the extraction efficiency of the process is in the range of 95-100% In another embodiment of the present invention, the purity of extracted biomolecules is in the range of 90-95%.

In an embodiment of the present invention, the biomolecules are selected from lutein, astaxanthin, colchicines, citral, citronellal and other important flavors and fragrances

In a preferred embodiment of the present invention, thebiomolecule is lutein.

In another embodiment of the present invention, the biomass is selected from yellow and brownish flower like marigold, maize, glory lily, algae like Haematococcus pluvialis, Chlorella zofingiensis, Chlorococcum, and Phaffiarhodozyma, some plants like lemon myrtle, Litseacitrata, Litseacubeba, lemongrass, lemon-tea-tree, Ocimum gratissimum, Linder acitriodora, Calypranthes parriculata, petitgrain, lemon verbena, lemon ironbark, lemon balm, lime, lemon and orange, ashwagandha etc

In still another embodiment of the present invention, the biomass including yellow and brownish flowers like marigold, maize, Colchicum autumnale (autumn crocus) are used for the extraction of lutein.

In a preferred embodiment of the present invention, the biomass used for extracting lutein is a marigold.

In still another embodiment of the present invention, the biomass glory lily is used for the extraction of Colchicine.

In yet another embodiment of the present invention, the biomass algae-like Haematococcus Pluvialis, Chlorella zofingiensis, Chlorococcun, and Phaffiarhodozyma are used for the extraction of astaxanthin.

In still another embodiment of the present invention, the bio mass plants like lemon myrtle, Litseacitrata, Litseacubeba, lemon grass, lemon tea-tree, Ocimum gratissimum, Linder acitriodora, Calypranthes parriculata, petitgrain, lemon verbena, lemon ironbark, lemon balm, lime, lemon and orange are used for extraction of citral and citronellal.

In an embodiment of the present invention, the ultrasonic process of extracting lutein pigments from marigold is done using an extraction apparatus that requires at least two repeating units of reactor set-up.

In a preferred embodiment of the present invention, the extraction apparatus is a reactor.

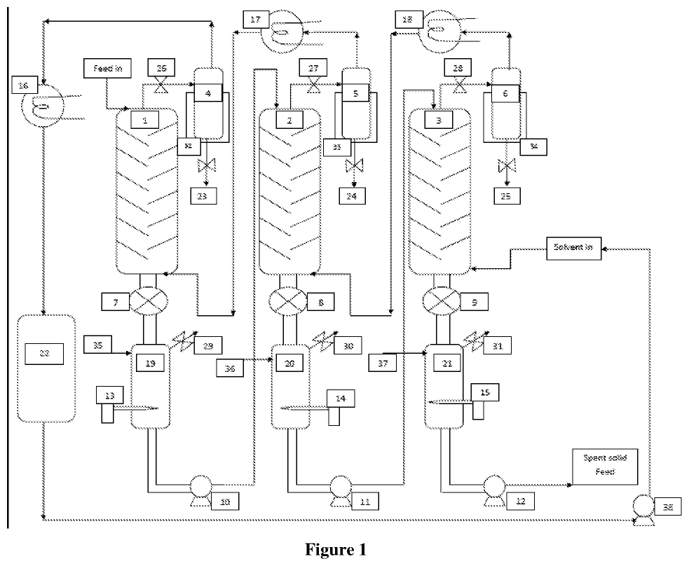

In another embodiment of the present invention, the reactor comprises:
a) Extractor (1-3)
b) Phase separator (4-6)
c) Rotary valve (7-9)
d) Pump (10-12)
e) Cavitation Probe (13-15)
f) Condenser (16-18)
g) Interstage processing Vessel (19-21)
h) Solvent Feed vessel (22)
i) Product collectors (23-25)
j) Pressure regulating valve (26-28)
k) Vent valves (29-31)
l) Heating Jacket (32-34)
m) Co-solvent (35-37)
n) Pump (38)
o) Feed inlet (39)
p) Solvent (40)
q) Spent solidFeed (41)
   The reactor set up of the present invention is depicted in FIG. 1. The reactor according to the embodiments of the present invention includes extractors (1-3) for continuous countercurrent extraction units designed to offer maximum extraction efficiency, designed to establish optimum solute and solvent contact; phase separators (4-6) to provide the required latent heat of evaporation to separate the solvent from extracted products; rotary valves (7-9) used to remove spent solid after the extraction of biomolecules; these valves are offering a continuous mode of operation for the entire process.

In addition, the reactor according to the embodiments of the present invention may further include pumps (10-12) to pump the interstage bio-mass and co-solvent into the extraction unit at the desirable pressure and flow rate; cavitation probes (13 and 14) to induce the desired ultrasonic cavitation effect into the interstage collection vessels processing tanks, which helps to break down the bimolecular cell wall; condensers (16 to 18) to condense and recycle the solvent; interstage processing vessels (19 to 21) to collect the under processing solid biomass, in these vessels, biomass is mixed with co-solvents, and it is treated under the cavitation inception.

The reactor according to the embodiments of the present invention may further include solvent feed vessel (22) to store feed solvent; product collectors (23 to 25) to collect product; pressure regulating valves (26 to 28) to maintain the desired pressure into the extractor units; vent valves (29 to 31) to vent the excess pressure to ensure the safe operation of the unit; heating jackets (32 to 34) to provide the required sensible heat to the solvent; pump (38) to feed solvent; solid biomass inlet (39) for the extraction; solvent inlet (40); spent solid feed (41) to outspent solid after complete extraction.

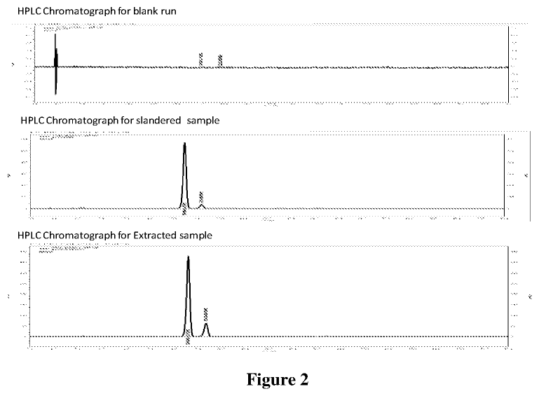

FIG. 2 represents the lutein HPLC Analysis Chromatograph. HPLC chromatography shows that the extracted lutein sample doesn't have any impurity peak and the purity profile is matching with the standard 97% commercial lutein sample.

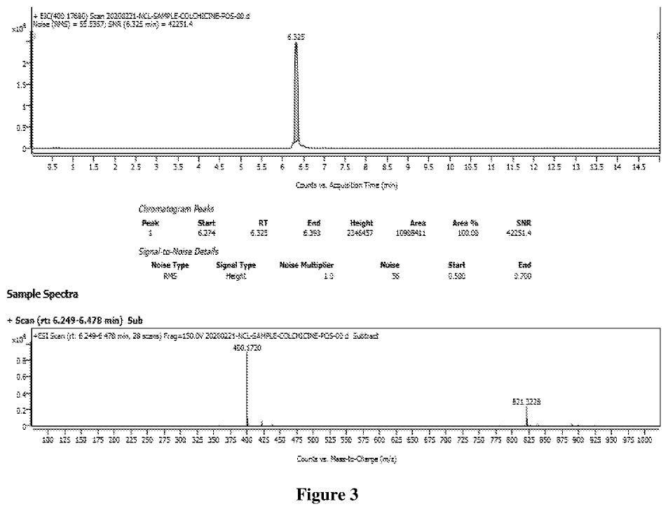

FIG. 3 represents colchicine analysis HPLC-MS Chromatograph. HPLC chromatography shows that the extracted colchicine sample does not have any impurity peak and the mass of the extracted colchicine is matching with pure colchicine's molecular mass that is 400.

### EXAMPLES

The following examples, which include preferred embodiments, will serve to illustrate the practice of this invention, it being understood that the particulars shown are by way of example and for purpose of illustrative discussion of preferred embodiments of the invention.

### Example 1: The Continuous Counter Current Sono-Chemically Controlled Extraction of Lutein from Marigold Flower

Marigold flower petals are collected and dried under a vacuum. The dried biomass is fed from the top hopper of the first extractor (1) at the feed rate of 1 kg/hr. At the same time, the solvent was pumped into the first extractor (1) counter currently through the second extractor (2) and the third extractor (3). The retention time of the solvent was kept for 15 min inside each extractor. The spent solid biomass from the first extractor (1) was mixed with co-solvent kept for controlled ultrasonic cavitation at 15 kHz for 5 min and then the slurry is pumped into the second extractor (2) where it interacted with flowing solvent. The spent solids from the second extractor (2) were mixed with co-solvent kept for controlled ultrasonic cavitation at 20 kHz for 5 min and then the slurry is pumped into the third extractor (3) where it interacted with flowing solvent. The spent solids from the third extractor (3) are taken out like spent biomass after extraction. The extracted lutein is collected from product collection vessels from the top of extractors 1, 2, and 3. Extractor 1 was operated at 45 degrees and 25 bar, extractor 2 was operated at 40 degrees and 30 bar, extractor 3 was operated at 35 degrees and 35 bar.

The extracted lutein samples, i.e. the spent biomass were analyzed for the calculation of extraction efficiency, and lutein purity was calculated using High-performance liquid chromatography, LC-MS.

Results:

The extraction efficiency was estimated as 100% and the purity of extracted lutein was found 95%. The same setup was also used for the extraction of Astaxynthin from algae Haematococcus pluvialis biomass and extraction of colchicines from the autumn crocus, where extraction efficiency of both the biomass was calculated as 100%.

### Advantages of the Invention

The present process is eco-friendly, cost-effective, easy solvent recovery, and requires comparatively less temperature, pressure and short reaction duration.
Sono-chemically derived radicals help for the extraction of the desired molecule without their decomposition.
This is the cost-effective and continuous process for the extraction of a molecule at mild temperature and pressure conditions.
Products are obtained with 100% extraction efficiency and 95% purity.

1. A continuous process of extraction of biomolecules from biomass by using an extraction apparatus comprising:
(a) introducing biomass into the first extractor, from the top inlet hopper followed by interacting the bio-mass with the extraction solvent, coming from the bottom of the extractor wherein the extraction solvent dissolves the biomass's valuable biomolecules;
(b) collecting the extracted biomolecules at the first phase separator and recycling the extraction solvent to the feed tank;
(c) treating the spent solids from the first extractor with co-solvent in the first interstage processing vessel in the influence of ultrasonic cavitation;
(d) pumping the partially processed biomass into the second extractor from the top inlet followed by interacting with extraction solvent which is coming from the bottom of the extractor wherein the extraction solvent dissolves the valuable biomolecules from the bio-mass;
(e) collecting the extracted biomolecules at the second phase separator, and the extraction solvent is condensed and sent to the first extractor;
(f) treating the spent solids from the second extractor with co-solvent in the second interstage processing vessel in the influence of ultrasonic cavitation;
(g) pumping the partially processed biomass from the second extractor into the third extractor from the top inlet followed by interacting with the extraction solvent, coming from the bottom of the third extractor wherein the extraction solvent dissolves the biomass's valuable biomolecules and
(h) collecting the extracted biomolecules at the third phase separator, and the extraction solvent is condensed and sent to the second extractor;
wherein, the spent solids from the third extractor are treated as spent solid mass.

2. The continuous process as claimed in claim 1, wherein temperature of the extraction process is in the range of 20-45° C.

3. The continuous process as claimed in claim 1, wherein operation pressure required for the extractor 3, extractor 2, and extractor 1 are in the range of 30-40 bar, 25-30 bar, and 15-25 bar respectively.

4. The continuous process as claimed in claim 1, wherein extraction efficiency of the process is in the range of 95-100%.

5. The continuous process as claimed in claim 1, wherein the purity of the extracted biomolecules is in the range of 90-95%.

6. The continuous process as claimed in claim 1, wherein the biomolecules are selected from but not limited to lutein, astaxanthin, colchicines, citral, or citronellal.

7. The continuous process as claimed in claim 2, wherein the biomolecule is lutein.

8. The continuous process as claimed in claim 1, wherein the biomass is selected from but not limited to marigold, maize, glory lily, Haematococcus pluvialis, Chlorella zofingiensis, Chlorococcum, Phaffiarhodozyna, lemon myrtle, Litseacitrata, Litseacubeba, lemongrass, lemon-tea-tree, Ocimum gratissimum, Linder acitriodora, Calypranthes parriculata, petitgrain, lemon verbena, lemon ironbark, lemon balm, lime, lemon, or orange.

9. The continuous process as claimed in claim 1, wherein the biomass is marigold.

10. An extraction apparatus for extraction of biomolecules from biomass comprising:
a) extractors including a first extractor, a second reactor and a third reactor to extract biomolecules from the biomass when the biomass interacts with an extraction solvent;
b) phase separators including a first phase separator, a second phase separator, and a third phase separator to provide a required latent heat of evaporation to separate the solvent from extracted products;
c) interstage processing vessels including a first interstage processing vessel, a second interstage processing vessel, and a third interstage processing vessel to collect the under processed solid biomass;
d) cavitation probes including a first cavitation probe, a second cavitation probe, and a third cavitation probe to induce the desired ultrasonic cavitation effect into the interstage processing vessels respectively; and
e) condensers including a first condenser, a second condenser, and a third condenser to condense and recycle the solvent.
